(19) United States
(12) Patent Application Publication
(10) Pub. No.: US 2009/0312237 A1
(43) Pub. Date: Dec. 17, 2009

(54) REGULATING FAT AND CARBOHYDRATE OXIDATION IN MUSCLE TISSUE COMPRISING CONTROLLING GARNITINE CONTENT IN MUSCLE

(75) Inventors: Paul Leonard Greenhaff, Nottingham (GB); Dumitru Constantin-Teodosiu, Nottingham (GB)

Correspondence Address: BROOKS KUSHMAN P.C., 1000 TOWN CENTER, TWENTY-SECOND FLOOR, SOUTHFIELD, MI, 48075, US

(73) Assignee: THE UNIVERSITY OF NOTTINGHAM, Nottingham (GB)

(21) Appl. No.: 12/295,973

(22) PCT Filed: Apr. 2, 2007
(86) PCT No.: PCT/GB2007/001209

§ 371 Date: Mar. 3, 2009

(30) Foreign Application Priority Data
Apr. 5, 2006 (GB) 0606864.7

(51) Int. Cl. A61K 38/28; A61K 31/205; A61K 31/70; A61K 38/02

(52) U.S. Cl. 514/4; 514/561; 514/23; 514/2

(57) ABSTRACT

Methods, compositions and substances for regulating fat and carbohydrate oxidation in the muscle tissue of human and/or animal bodies, which involve controlling the carnitine content of the muscle tissue. Regulating carbohydrate and fat oxidation, and particularly increasing the level of fat oxidation in accordance with the present invention, by providing substances compositions and methodologies in accordance with the present invention, enable medicaments to be manufactured and methodologies employed that find use in the treatment of disorders and conditions of the human and/or animal body caused by impairment of the usual level of fat oxidation, including obesity and type 2 diabetes.

The present invention relates to the regulation of fat and carbohydrate oxidation in muscle tissue and particularly, but not exclusively, to compositions, substances and methods of regulating and controlling fat and carbohydrate oxidation in a human and/or animal body. Both fat oxidation and carbohydrate oxidation within the muscle tissue of humans and animals are very important biochemical processes and, when not functioning properly, can affect the proper functioning of the muscle tissue, which in turn can lead to disorders of the tissue or body. For instance, when the normal daily level of fat oxidation is chronically impaired, this is believed to contribute to various conditions and disorders of the body such as obesity and type 2 diabetes. This impairment of fat oxidation can also affect the performance and function of a body during periods of metabolic stress, such as during exercise when there is an increase in energy demand of the musculature.

According to the present invention there is provided a method of regulating fat oxidation in muscle tissue of a human and/or animal body, the method comprising controlling the carnitine content of the muscle tissue.

The term carnitine is used in this specification to refer to naturally occurring carnitine, functional derivatives, functional equivalents and carnitine analogues.

Preferably the method enables the level of fat oxidation to be selectively increased, preferably by controlled increase in the level of carnitine entering the muscle tissue.

Preferably, the carnitine content of the muscle tissue is controlled by controlling the level of carnitine uptake from the bloodstream of the body into the muscle tissue.

Preferably, the carnitine concentration in the blood stream is controlled, preferably by the controlled ingestion or other administration of carnitine to the body.

Preferably, the uptake of carnitine into the muscle tissue is controlled by controlling the activity of a carnitine transport protein involved in the transportation of carnitine into muscle tissue.

Preferably, the uptake of carnitine is controlled, preferably further controlled, by controlling the level of insulin in the bloodstream, which may be done by controlled administration of insulin or functional equivalents thereof to the body and/or controlled administration or ingestion of substances to stimulate the production of insulin by the body, such as carbohydrate, for example, sugar and/or amino acid(s) and/or protein(s), to the body.

The uptake of carnitine may be controlled by controlling the level and/or activity of insulin in the body.

According to a second aspect of the present invention there is provided a method of regulating carbohydrate oxidation and storage in muscle tissue of a human and/or animal body, the method comprising controlling the carnitine content of the muscle tissue.

The method preferably enables the level of carbohydrate oxidation to be selectively decreased, and consequently the level of carbohydrate storage to be increased, by controllably increasing the level of carnitine entering the muscle tissue.

The method may be as described in paragraphs five to nine above.

According to a third aspect of the present invention there is provided a composition for use in the regulation of fat oxidation in muscle tissue of a human and/or animal body, the composition comprising carnitine and an agent to promote the uptake of carnitine into the muscle tissue.

The agent may comprise one or more of insulin, a functional derivative or analogue of insulin, carbohydrate, protein, amino acid and any other substance that acts to increase the level of insulin and/or insulin activity in the body either directly or by stimulating the production of insulin by the body.

The composition may be ingestible and may be in the form of a liquid, solid, tablet, pellet, powder.

Alternatively, the composition may be suitable for administration to a body by way of injection or other suitable means.

Preferably the composition acts to increase the amount of carnitine available to the tissue.

According to a fourth aspect of the present invention there is provided a composition for use in the regulation of carbohydrate oxidation and storage in muscle tissue of a human and/or animal body, the composition comprising carnitine and an agent to promote the uptake of carnitine into the muscle tissue.

The composition may be as described in any of paragraphs fourteen to seventeen above.

According to a fifth aspect of the present invention there is provided a substance for use in the manufacture of a medicament for the treatment of obesity in a human and/or animal body, the substance comprising carnitine and an agent to promote the uptake of carnitine into the muscle tissue of the body.

Preferably the substance acts to increase the carnitine content of the muscle tissue.

The substance may comprise a composition as described in any of paragraphs thirteen to nineteen above.

According to a sixth aspect of the present invention there is a provided a substance for the use in the manufacture of a medicament for the treatment of diabetes, the substance comprising carnitine and an agent to promote the uptake of carnitine into the muscle tissue.

Preferably the substance acts to increase the carnitine content of the muscle tissue.

The substance may be particularly useful in the manufacture of a medicament for the treatment of type 2 diabetes and may comprise a composition as described in any of paragraphs thirteen to nineteen above.

According to a seventh aspect of the present invention there is provided a substance for use in the manufacture of a medicament for the treatment of disorders in the animal or human body caused by deficiencies in fat oxidation in muscle tissue, the substance comprising carnitine and an agent to promote the uptake of carnitine into the muscle tissue.

The substance may comprise a composition as described in any of paragraphs thirteen to nineteen above.

According to an eighth aspect of the present invention there is provided a food supplement comprising a composition as described in any of paragraphs thirteen to nineteen above.

According to a ninth aspect of the present invention there is provided a method of treating disorders or conditions of the human and/or animal body caused by deficiencies in fat oxidation in muscle tissue, the method comprising regulating the carnitine content of the muscle tissue.

The method may be as described in any of paragraphs two to nine above.

According to a tenth aspect of the present invention there is provided a method of treating disorders or conditions of the human and/or animal body caused by deficiencies in carbohydrate storage in muscle tissue, the method comprising regulating carbohydrate oxidation and thereby storage in the muscle tissue.

The method may be as described in any of paragraphs ten to twelve above.

According to an eleventh aspect of the present invention there is provided a method of treating obesity of a human or animal body, the method comprising regulating the carnitine content of the muscle tissues of the body.

The method may be as described in any of paragraphs two to twelve above.

According to a twelfth aspect of the present invention there is provided a method of treating diabetes in a human or animal body, the method comprising regulating the carnitine content of the muscle tissue in the body.

Preferably the method is for treating type 2 diabetes.

The method may be described in any of paragraphs two to twelve above.

Preferred embodiments of the present invention will now be described by way of example only, with reference to the accompanying drawings, in which:

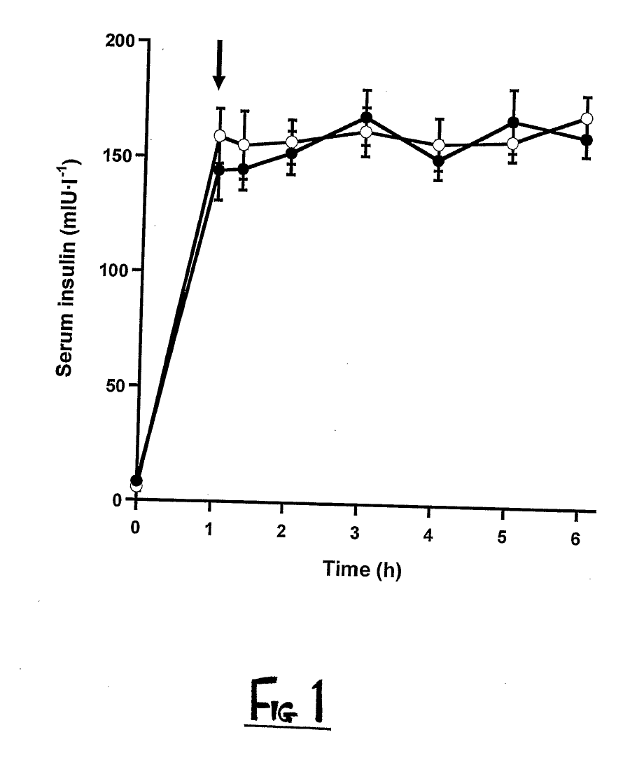

FIG. 1 shows a plot of serum insulin concentration over the course of 6 h of intravenous insulin infusion at a rate of 105 mlU·m⁻²·min⁻¹ during the CON (∘) and CARN () infusion visits. The arrow indicates the commencement of the saline (CON) or L-carnitine infusion. Values are means±SEM (n=7).

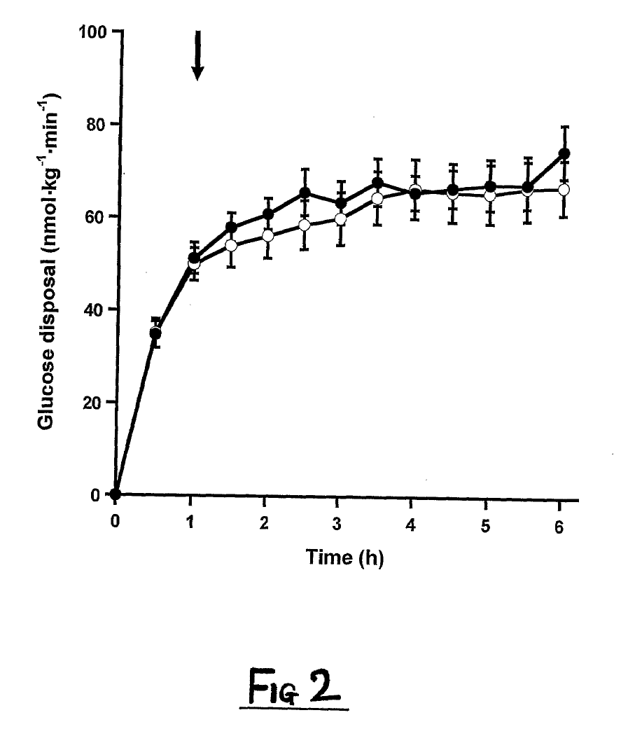

FIG. 2 shows a plot of whole body glucose disposal over the course of 6 h of intravenous insulin infusion at a rate of 105 mlU·m⁻²·min⁻¹ during the CON (∘) and CARN () infusion visits. The arrow indicates the commencement of the saline (CON) or L-carnitine infusion. Values are means±SEM (n=7).

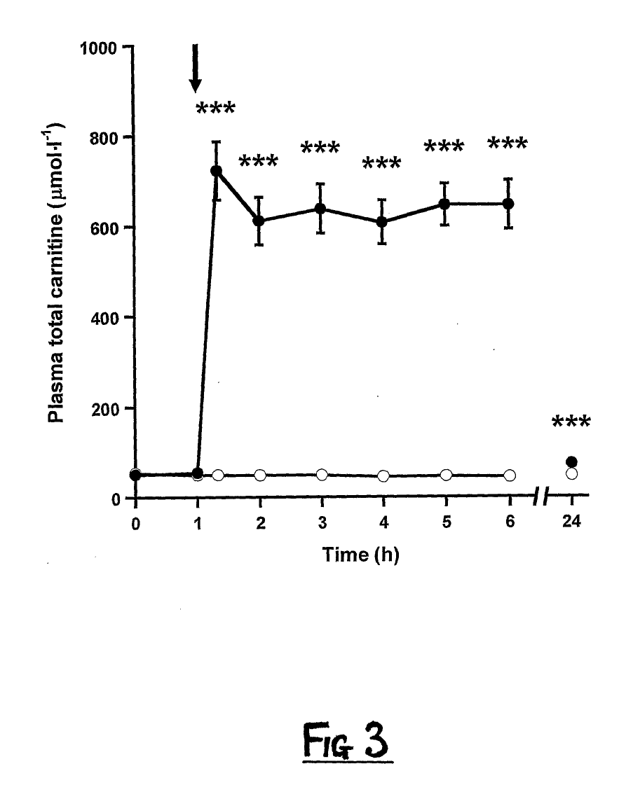

FIG. 3 shows a plot of plasma total carnitine concentration over the course of 5 h of intravenous saline (CON; ∘) and L-carnitine (CARN; ) infusion combined with 6 h intravenous insulin infusion at a rate of 105 mlU·m⁻²·min⁻¹. The arrow indicates the commencement of the saline or L-carnitine infusion. Values are means±SEM (n=7). ***P<0.001, significantly greater than the corresponding CON value.

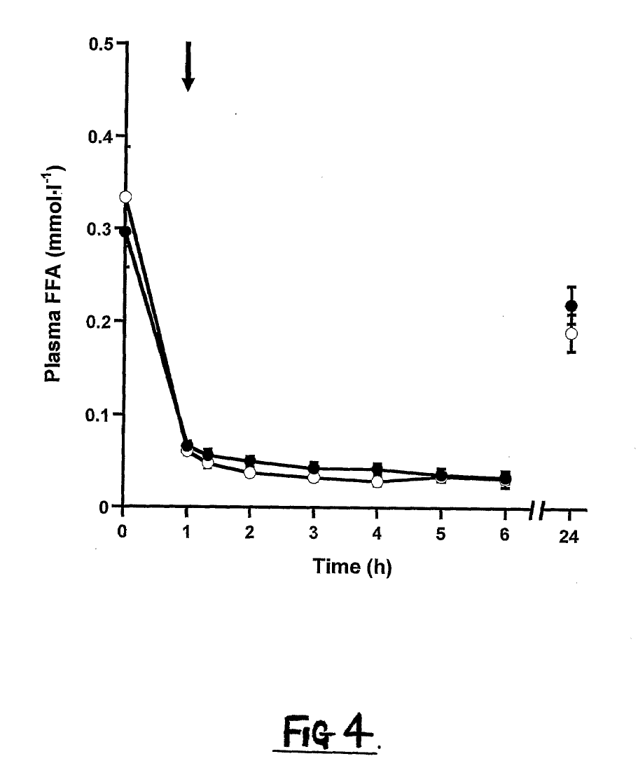

FIG. 4 shows a plot of plasma FFA concentration over the course of 5 h of intravenous saline (CON; ∘) and L-carnitine (CARN; ) infusion combined with 6 h intravenous insulin infusion at a rate of 105 mlU·m⁻²·min⁻¹. The arrow indicates the commencement of the saline or L-carnitine infusion. Values are means±SEM (n=7).

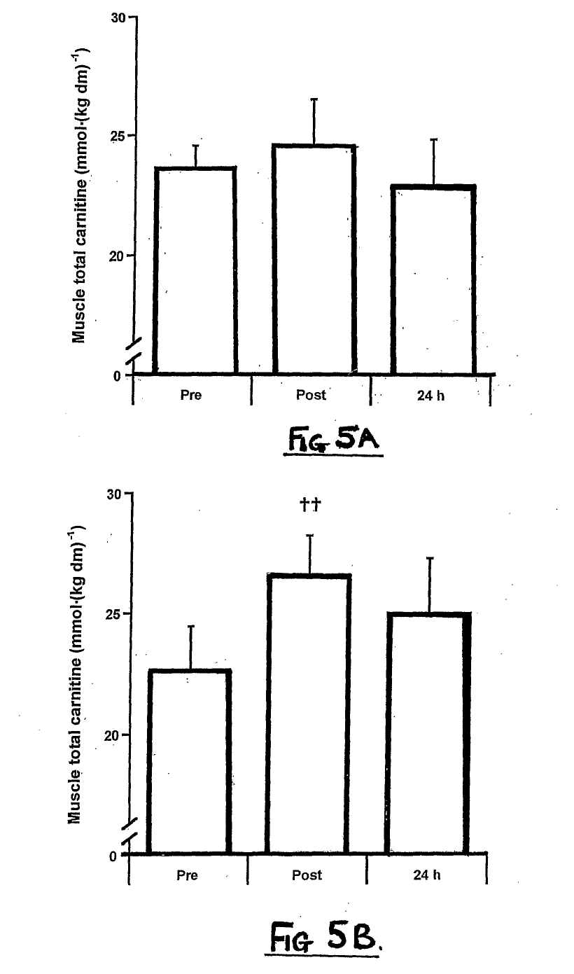

FIG. 5A shows a plot of muscle total carnitine (TC) content before and after 5 h of intravenous saline infusion accompanied by a euglycaemic hyperinsulinaemic clamp, and 24 h after the commencement of the respective infusion visits. Values represent means±SEM (n=7). ^{††}P<0.01, greater than pre infusion value.

FIG. 5B shows a plot of muscle total carnitine (TC) content before and after 5 h of intravenous L-carnitine infusion accompanied by a euglycaemic hyperinsulinaemic clamp and 24 h after the commencement of the respective infusion visits. Values represent means±SEM (n=7). ^{††}P<0.01, greater than pre infusion value.

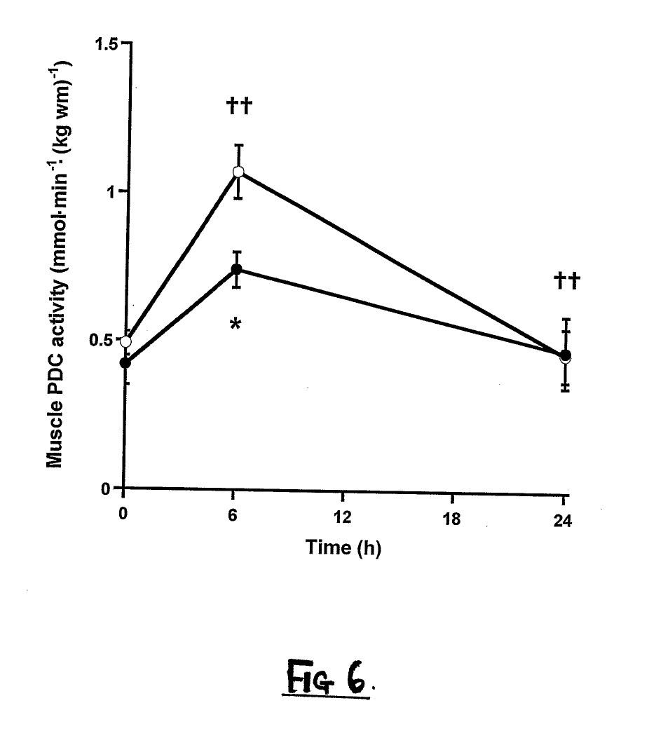

FIG. 6 shows a plot of muscle pyruvate dehydrogenase complex (PDC) activity before and after 5 h of intravenous saline (CON; ∘) and L-carnitine (CARN; ) infusion accompanied by a euglycaemic hyperinsulinaemic clamp, and 24 h after the commencement of the respective infusion visits. Values represent means±SEM (n=7). ^{††}P<0.01, significantly greater than pre CON and CARN infusion value, and significantly less than post CON and CARN infusion value. *P<0.05, CARN significantly less than corresponding CON value.

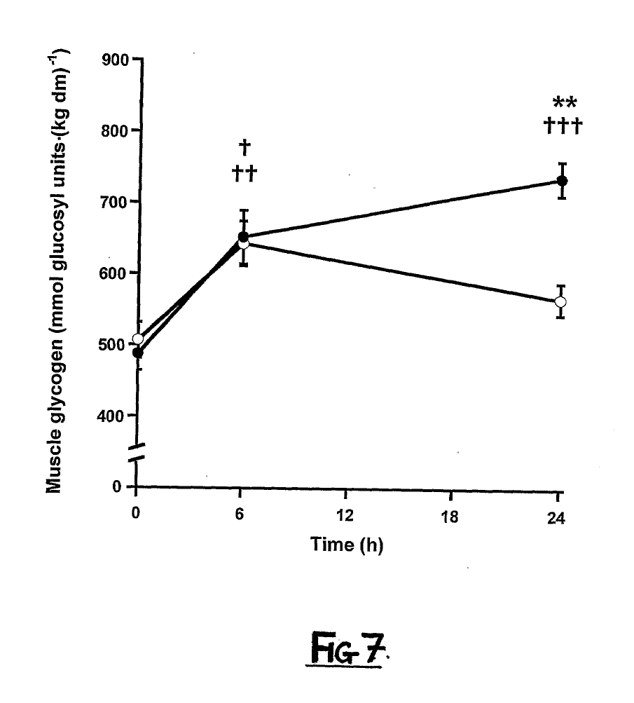

FIG. 7 shows a plot of muscle glycogen content before and after 5 h of intravenous saline (CON; ∘) or L-carnitine (CARN; ) infusion accompanied by a euglycaemic hyperinsulinaemic clamp, and 24 h after the commencement of the respective infusion visits. Values represent means±SEM (n=7). **P<0.01, CARN significantly greater than corresponding CON value.

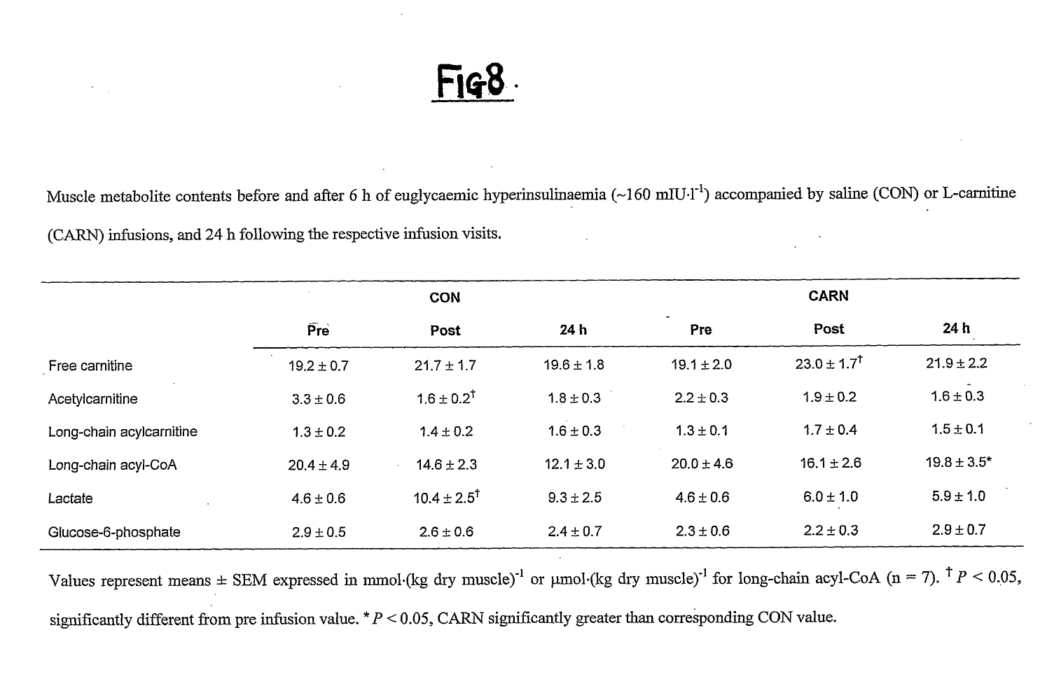

FIG. 8 in a table of muscle carnitine moieties before, immediately after and 24 h after the infusion visits.

Referring to the figures the invention provides methods, compositions and substances for regulating fat oxidation and carbohydrate oxidation and storage in the muscle tissue of human and/or animal bodies, which involve controlling the carnitine content of the muscle tissue.

Regulating carbohydrate and fat oxidation, and particularly increasing the level of fat oxidation in accordance with the present invention, by providing substances compositions and methodologies in accordance with the present invention, enable medicaments to be manufactured and methodologies employed that find use in the treatment of disorders and conditions of the human and/or animal body caused by impairment of the usual level of fat oxidation, including obesity and type 2 diabetes.

Below is a description of work done that revealed the novel development that by regulating the total carnitine content in muscle tissue the levels of fat oxidation and carbohydrate oxidation can be selectively controlled, enabling significant advances in the development of medicaments, substances, compositions and methodologies to help in the treatment of associated disorders and to facilitate muscle tissue function during conditions of exercise and similar.

Seven healthy, non-vegetarian young men (age 22.4±1.5 yr, body mass 84.3±5.0 kg, body mass index 26.1±1.6 kg·mM⁻²) participated in the study. Each subject reported to the laboratory at 8 am on two occasions, separated by a 2-week “wash out” period, and voided their bladder. All subjects had abstained from carnitine containing foods, alcohol, and strenuous exercise for the previous 24 hours. On arrival, subjects were asked to rest in a supine position on a bed while a cannula was inserted retrogradely in to a superficial vein on the dorsal surface of the non-dominant hand. This hand was kept in a hand-warming unit (air temperature 50-55° C.) to arterialize the venous drainage of the hand, and a saline drip was attached to keep the cannula patent. A second cannula was placed in an antecubital vein in the non-dominant forearm for the infusion of insulin and glucose, and a third cannula was inserted into an antecubital vein in the opposite arm for infusion of L-carnitine.

On each experimental visit, a 6 hour (h) euglycaemic hyperinsulinaemic insulin (human Actrapid, Novo Nordisk, Denmark) clamp was performed, whilst maintaining a fasting blood glucose concentration of 4.47±0.01 mmol·l⁻¹. The insulin clamp began at t=0 at a rate of 105 mlU·m⁻²·min⁻¹ with the aim of achieving steady state hyperinsulinaemic (˜150 mlU·l⁻¹) serum insulin concentration throughout each visit. Following a 1 hour equilibration period, a 5 hour intravenous infusion of 60 mM L-carnitine (CARN) (Lonza Ltd, Basel, Switzerland) or the equivalent volume of saline (CON) began, in randomised manner, in conjunction with the insulin clamp. At the onset of L-carnitine infusion a bolus dose of 15 mg·kg⁻¹ was administered over 10 minutes (min) in order to rapidly reach a supraphysiological plasma concentration of ˜500 μmol·l⁻¹. This was followed by a constant infusion at 10 mg·kg⁻¹·h⁻¹ for the next 290 min to maintain hypercarnitinaemia. At t=6 h, the insulin and L-carnitine infusions were stopped, whereas the glucose infusion was continued for approximately 80 min in order to stabilise blood glucose concentration. During this time, on each visit, subjects were fed the same standardised, carnitine-free meal. The meal had an energy content of approximately 1500 kcal (55% carbohydrate, 35% fat, and 10% protein), and a total carbohydrate content of 220 g (35% of which were sugars).

Thereafter, subjects were free to leave the laboratory once their blood glucose concentration was stable. Any food/drink from the meal that was not consumed before subjects left the laboratory was consumed that evening before 10 pm (usually consisting of a chocolate bar and a beverage high in carbohydrate), with the time and amount being noted and repeated on the following experimental visit. Subjects then returned to the laboratory at 8 am the following morning in a fasted state from the previous evening.

During each experimental visit, 1 ml of arterialized venous (a-v) blood was obtained every 5 min for monitoring blood glucose concentration (YSI 2300 STATplus, Yellow Springs Instruments, Ohio). In addition, 5 ml of a-v blood were obtained every hour (and at 80 min) for 6 h, and at 24 h the following morning. Two ml of this blood were collected into lithium heparin containers and, after centrifugation, the plasma was removed and immediately frozen in liquid nitrogen. These samples were then stored at −80° C. and analyzed at a later date for free fatty acid (FFA) concentration, using an enzymatic-colorimetric assay kit (NEFA C kit, WAKO Chemicals, Germany), and total carnitine (TC) concentration, using the radioenzymatic assay described previously by Cederblad et al (1982). The remaining blood was allowed to clot, and, after centrifugation, the serum was stored frozen at −80° C. Insulin was measured in these samples at a later date with a radioammunoassy kit (Coat-a-Count Insulin, DPC, Ca, USA).

Muscle biopsy samples were obtained from the vastus lateralis muscle immediately before and after each insulin clamp, and the following morning, using the percutaneous needle biopsy technique, and were snap frozen in liquid nitrogen less than 5 seconds after removal from the limb. One portion of the sample was subsequently freeze-dried and stored at −80° C., and the remainder was stored “wet” in liquid nitrogen. After removal of visible blood and connective tissue, the freeze-dried muscle samples were powdered, and free carnitine (FC), acetylcarnitine (AC), long-chain acylcarnitine (LCAC), and long-chain acyl-CoA (LCACoA) contents were determined radioenzymatically using a modified version of the radioenzymatic method of Cederblad et al (1990). Values were subsequently summed in order to calculate muscle TC. To reduce the variance in non-muscle constituents, muscle carnitine content was adjusted for the highest total creatine content from each pair of samples. Total creatine was calculated as the sum of free creatine and phosphocreatine content determined spectrophotometrically using the method of Harris et al (1974). Muscle glycogen, glucose-6-phosphate (G-6-P), and lactate content was also determined using a modified version of the spectrophotometric method of Harris et al (1974).

The remainder of the frozen muscle was used to determine PDC activity as previously described (Constantin-Teodosiu et al, 1991). Briefly, the activity of PDC in its dephosphorylated active form (PDCₐ) was assayed in a buffer containing NaF and dichloroacetate (DCA) and was expressed as rate of acetyl-CoA formation (mmol·min¹·(kg wet muscle)⁻¹ or nmol·min¹·(mg protein)⁻¹) at 37° C. Protein concentrations were determined using the method of Peterson (1977).

A two-way ANOVA (time and treatment effects; GraphPad Prism 4.02, GraphPad Software Inc, CA) was performed to locate differences in serum insulin, plasma carnitine, plasma FFA, and blood glucose concentrations as well as muscle PDC activity and carnitine, long-chain acyl-CoA, glycogen, lactate, and glucose-6-phosphate content. When a significant main effect was detected, data were further analysed with Student's paired t tests using the Bonferroni correction. Statistical significance was declared at P<0.05, and all the values presented in text, Tables, and Figures are means±standard error of the mean (SEM).

### Results

### Serum Insulin and Glucose Disposal

Serum insulin concentration profiles are presented in FIG. 1. Following the 60 min equilibration period, the 105 mlU·m⁻²·min⁻¹ euglycaemic hyperinsulinaemic clamps produced similar steady state serum insulin concentrations of 160.1±1.9 and 155.8±3.9 mlU·l⁻¹, during the saline (CON) and L-carnitine (CARN) infusions, resulting in whole body glucose disposal values of 300.2±19.3 and 316.8±15.0 g over the 6 h, respectively (FIG. 2). A rapid increase in whole body glucose disposal was observed during the first hour (equilibration period) of the euglycaemic hyperinsulinaemic clamps, followed by a more gradual increase for the remaining 5 h. Whole body glucose disposal was also similar between the 80 min recovery periods following the two clamps of 32.5±4.1 and 37.2±3.5 g for CON and CARN, respectively.

### Plasma Carnitine

The plasma total carnitine TC profiles over the course of the CON and CARN infusion visits during the two hyperinsulinaemic clamps are illustrated in FIG. 3. From similar basal plasma TC concentrations of 53.8±4.3 and 50.5±4.6 μmol·l⁻¹, the saline infusion had no effect on plasma TC concentration, whereas the bolus 15 mg·kg⁻¹ L-carnitine infusion (indicated by the arrow) produced a mean peak plasma TC concentration of 723.0±63.9 μmol·l⁻¹, which remained elevated above 600 μmol·l⁻¹ throughout the CARN visit, and was greater than during CON at every time point (P<0.001). Plasma TC concentration during CARN was also greater than CON the following morning after 24 h (47.7±3.8 vs. 72.9±5.3 μmol·l⁻¹, for CON and CARN, respectively; P<0.001).

### Plasma Free Fatty Acids (FFA)

FIG. 4 illustrates plasma FFA concentration over the course of the CON or CARN infusion visit during each hyperinsulinaemic clamp. From similar basal concentrations of 0.33±0.05 and 0.30±0.04 mmol·l⁻¹, plasma FFA concentration decreased following the commencement of the insulin clamp, and was maintained at 0.039±0.002 and 0.046±0.01 mmol·l⁻¹ during the CON and CARN visit, respectively. By 24 h, plasma FFA concentration was 0.22±0.02 and 0.19±0.02 mmol·l⁻¹, during the CON and CARN visit, respectively. There were no significant differences between each visit.

### Muscle Carnitine

Skeletal muscle total carnitine (TC) data during the CON visit are presented in FIG. 5A. Skeletal muscle TC content was unchanged following 5 h of saline infusion in conjunction with hyperinsulinaemia (23.7±0.9 vs. 24.7±1.8 mmol·(kg dry muscle)⁻¹), and was the same as the basal state the following morning (23.0±1.9 mmol·(kg dm)⁻¹). However, 5 h of L-carnitine infusion during hyperinsulinaemia (CARN visit) increased skeletal muscle TC by 15% (22.5±2.0 vs. 26.6±1.6 mmol·(kg dm)⁻¹, P<0.01), which remained elevated over night, although it was not significantly different from the pre infusion value (25.1±2.2 mmol·(kg dm)⁻¹; FIG. 5B).

Skeletal muscle carnitine moieties before, immediately after, and 24 h after the infusion visits are presented in FIG. 8. There was a trend for an increase (12%) in muscle free carnitine (FC) content following the CON infusion visit, which was paralleled by a corresponding decrease in acetyl carnitine (AC) (P<0.05). Muscle FC increased by 17% during the CARN infusion visit (P<0.05), retuning to basal overnight, whereas no change was observed in AC. Skeletal muscle long chain acyl carnitine was unchanged following both infusion visits.

### Pyruvate Dehydrogenase Complex (PDC) Activity

Muscle PDC activity increased during the euglycaemic hyperinsulinaemic clamp from 0.49±0.04 to 1.07±0.09 mmol·min⁻¹·(kg wm)⁻¹ (4.0±0.6 to 7.1±1.2 nmol·min⁻¹·(mg protein)⁻¹; P<0.01) during the CON visit and from 0.43±0.07 to 0.74±0.06 mmol·min⁻¹·(kg wm)⁻¹ (4.2±1.6 to 4.1±0.9 nmol·min⁻¹·(mg protein)⁻¹; P<0.01) during the CARN visit (FIG. 6). However, the increase in PDC activity following the CARN infusion visit was not as pronounced as following the CON infusion visit, such that post infusion PDC activity was 31% less than CON (P<0.05). Twenty-four hours after the commencement of the insulin infusion, PDC activity had returned to its resting value of 0.46±0.09 (P<0.01) and 0.47±0.12 mmol·min⁻¹·(kg wm)⁻¹·(3.6±1.0 and 3.9±0.8 nmol·min⁻¹·(mg protein)⁻¹) for the CON and CARN visit, respectively.

### Muscle Metabolites

Muscle glycogen content increased during each euglycaemic hyperinsulinaemic clamp, from the resting content of 506±25 and 487±23 mmol·(kg dm)⁻¹, to post infusion values of 642±31 (P<0.05) and 651±37 mmol·(kg dm)⁻¹ (P<0.01) during CON and CARN visits, respectively (FIG. 7). Twenty-four hours after the commencement of the insulin infusion during the CON visit, muscle glycogen had not changed (567±22 mmol·(kg dm)⁻¹), whereas following the CARN visit, muscle glycogen content increased to 736±24 mmol·(kg dm)⁻¹, which was significantly greater than the pre infusion value (P<0.001) and the corresponding 24 h CON value (P<0.01).

Skeletal muscle lactate content, presented in FIG. 8, was increased 56% (P<0.05) following the CON infusion visit, but unchanged following the CARN infusion visit. Twenty-four hours after the commencement of the saline infusion, muscle lactate content returned to basal. No significant differences were observed in skeletal muscle glucose-6-phosphate content during each visit (FIG. 8). Skeletal muscle long chain acyl-CoA (LCACoA) content tended to decrease following the CON and CARN infusion visits (FIG. 8). However, whereas muscle LCACoA remained lower (P=0.08) the following morning after the cessation of the hyperinsulinaemic clamp in the CON visit, muscle LCACoA content had returned to basal at the corresponding time point in the CARN visit such that it was 39% greater than CON (P<0.05).

The above study shows that a 15% increase in skeletal muscle carnitine content (FIG. 5B), achieved via intravenous L-carnitine infusion during a 6 hour euglycaemic hyperinsulinaemic clamp, resulted in a 30% decrease in PDC activity (FIG. 6) and a 40% decrease in muscle lactate content (FIG. 8). Furthermore, following an overnight fast, muscle glycogen (FIG. 7) and long-chain acyl-CoA (LCACOA; FIG. 8) content had increased by 30 and 40%, respectively, in the CARN group compared with CON. The total amount of glucose infused during the CON and CARN visits was approximately 330 and 350 g, respectively, and the subjects consumed the same diet following each visit, consisting of approximately 220 g of carbohydrate. Thus, the 250 g difference in whole body muscle glycogen content (assuming skeletal muscle contributes to 40% of total body mass) between the CON and CARN visits was not due to any difference in the amount of carbohydrate administered. This shows that increased muscle carnitine content inhibits carbohydrate oxidation at the level of PDC and glycolytic flux (decrease in lactate), thereby diverting muscle glucose uptake towards glycogen storage (non-oxidative glucose disposal).

An increase in non-oxidative glucose disposal, calculated indirectly as the difference between whole body glucose disposal and oxidation (measured by indirect calorimetry), during steady-state L-carnitine infusion in the presence of an elevated serum insulin concentration (˜75 mU·l⁻¹), has been previously reported in other human studies. However, the fate of the glucose was not determined in these experiments, nor were the mechanisms involved elucidated. The general assumption from the studies was that, based on reduced circulating pyruvate and increased plasma acetylcarnitine concentrations, L-carnitine infusion lowered the skeletal muscle acetyl-CoA/CoASH ratio resulting in an increase in muscle PDC activity. The acetyl-CoA subsequently formed from pyruvate was diverted to acetylcarnitine, rather than the TCA cycle, resulting in the observed increase in non-oxidative glucose disposal.

In contrast to this theory, it was demonstrated during the CARN visit of the present study that there is a decrease in PDC activity and a diversion of glucose uptake to glycogen storage.

Due to carnitine's role in long-chain fatty acid translocation into the mitochondrial matrix the reduction in PDC activation is believed to have been caused by a carnitine·mediated increase in skeletal muscle long-chain fatty acid oxidation via carnitine palmitoyl transferase 1 (CPT1) (possibly from intramuscular stores, as plasma FFA was not different between visits; FIG. 4). The decrease in PDC activity following the insulin clamp in the CARN trial of the present study was paralleled by a reduction in muscle lactate content and resulted in an accumulation of muscle glycogen overnight, conditions which are both consistent with the premise that carbohydrate oxidation was inhibited. Muscle LCACOA content also returned to basal overnight during the CARN visit (whereas it was still suppressed during the CON visit), which reflects an increase in fat oxidation.

The reciprocal relationship between carbohydrate and fat oxidation in skeletal muscle would suggest that the apparent decrease in carbohydrate flux observed was the result of, or resulted in, an increase in fat oxidation. These findings are of potentially major importance in the treatment of insulin-resistant states, such as obesity and type 2 diabetes, as both conditions are associated with an impaired ability of skeletal muscle to oxidize fat (fatty acids), both at rest and during exercise. Furthermore, reducing or preventing intramuscular lipid accumulation increases insulin sensitivity. In addition, a carnitine-mediated inhibition of carbohydrate oxidation (at the level of PDC) and an increase in muscle glycogen storage could also be of relevance in these conditions, as the inability of insulin to activate GS in obese individuals appears to precede the development of type 2 diabetes (Schalin-Jantti et al, 1992; Jensen et al, 2006). Increasing skeletal muscle fat oxidation in obesity and type 2 diabetes is important, particularly during exercise, as exercise combined with weight loss, rather than weight loss alone, enhances fasting skeletal muscle fat oxidation rates and improves insulin sensitivity in obese patients. Free carnitine availability could be responsible for the impairment of fat oxidation observed during exercise in obesity/type 2 diabetes. There is enhanced glucose utilization during exercise in obesity and type 2 diabetes, and it is feasible therefore that this could reduce muscle free carnitine content because of PDC flux being in excess of the rate of acetyl-CoA utilization by the TCA cycle, which would decrease fat oxidation. Accordingly, by increasing skeletal muscle free carnitine availability in accordance with the present invention, the inhibition of fat oxidation observed during exercise in these conditions could be alleviated.

In conclusion, increasing skeletal muscle carnitine content, via an insulin-mediated increase in muscle carnitine uptake, decreases PDC activity and muscle lactate content, and increases glycogen storage in conditions of high carbohydrate availability, by carnitine mediated increase in muscle fat oxidation as muscle LCACOA was increased. This is an important development in the regulation of muscle fat oxidation, particularly during exercise, when carnitine availability may limit fat oxidation, and in obesity and type 2 diabetes where fat oxidation is known to be impaired.

Various modification may be made without departing from the spirit and scope of the present invention. Reference to carnitine herein includes natural carnitine, functional derivatives and functional equivalents and analogues of carnitine.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A method of regulating fat oxidation in muscle tissue of a human and/or animal body, the method comprising controlling the carnitine content of the muscle tissue.

2. A method of regulating fat oxidation as claimed in claim 1, in which the method enables the level of fat oxidation to be selectively increased.

3. A method of regulating fat oxidation as claimed in claim 2, in which the level of fat oxidation is selectively increased by controllably increasing the level of carnitine entering the muscle tissue.

4. A method of regulating fat oxidation as claimed in claim 1, in which the carnitine content of the muscle tissue is controlled by controlling the level of carnitine uptake from the bloodstream of the body into the muscle tissue.

5. A method of regulating fat oxidation as claimed in claim 4, in which the carnitine concentration in the blood stream is controlled.

6. A method of regulating fat oxidation as claimed in claim 5, in which the carnitine concentration in the bloodstream is controlled by the controlled ingestion or other administration of carnitine to the body.

7. A method of regulating fat oxidation as claimed in claim 4, in which the uptake of carnitine into the muscle tissue is controlled by controlling the activity of a carnitine transport protein involved in the transportation of carnitine into muscle tissue.

8. A method of regulating fat oxidation as claimed in claim 4, in which the uptake of carnitine is controlled, preferably further controlled, by controlling the level of insulin in the bloodstream.

9. A method of regulating fat oxidation as claimed in claim 8, in which the level of insulin in the bloodstream is controlled by controlled administration of insulin or functional equivalents thereof to the body and/or controlled administration or ingestion of substances to stimulate the production of insulin by the body, such as carbohydrate, for example, sugar and/or amino acid(s) and/or protein(s), to the body.

10. A method of regulating fat oxidation as claimed in claim 4, in which the uptake of carnitine is controlled by controlling the level of insulin activity in the body.

11. A method of regulating carbohydrate oxidation and storage in muscle tissue of a human and/or animal body, the method comprising controlling the carnitine content of the muscle tissue.

12. A method of regulating carbohydrate oxidation as claimed in claim 11, in which the method enables the level of carbohydrate oxidation to be selectively decreased, and consequently the level of carbohydrate storage to be increased, by controllably increasing the level of carnitine entering the muscle tissue.

13. A method of regulating carbohydrate oxidation and storage in muscle tissue of a human and/or animal body, the method comprising controlling the carnitine content of the muscle tissue, in which the method comprises one or more of the steps of claim 4.

14. A composition for use in the regulation of fat oxidation in muscle tissue of a human and/or animal body, the composition comprising carnitine and an agent to promote the uptake of carnitine into the muscle tissue.

15. A composition as claimed in claim 14, in which the agent may comprise one or more of insulin, a functional derivative or analogue of insulin, carbohydrate, protein, amino acid and any other substance that acts to increase the level of insulin and/or insulin activity in the body either directly or by stimulating the production of insulin by the body.

16. A composition as claimed in claim 14 in which the composition may be ingestible.

17. A composition as claimed in claim 14, in which the composition is in the form of a liquid, solid, tablet, pellet or powder.

18. A composition as claimed in claim 14, in which the composition is suitable for administration to a body by way of injection or other suitable means.

19. A composition as claimed in claim 14, in which the composition acts to increase the amount of carnitine available to the tissue.

20. A composition for use in the regulation of carbohydrate oxidation and storage in muscle tissue of a human and/or animal body, the composition comprising carnitine and an agent to promote the uptake of carnitine into the muscle tissue.

21. A composition for use in the regulation of carbohydrate oxidation and storage in muscle tissue of a human and/or animal body, the composition comprising carnitine and an agent to promote the uptake of carnitine into the muscle tissue, the composition being as defined in claim 15.

22. A substance for use in the manufacture of a medicament for the treatment of obesity in a human and/or animal body, the substance comprising carnitine and an agent to promote the uptake of carnitine into the muscle tissue of the body.

23. A substance as claimed in claim 22, in which the substance acts to increase the carnitine content of the muscle tissue.

24. A substance for use in the manufacture of a medicament for the treatment of obesity in a human and/or animal body, the substance comprising carnitine and an agent to promote the uptake of carnitine into the muscle tissue of the body, in which the substance comprises a composition as defined in claim 14.

25. A substance for the use in the manufacture of a medicament for the treatment of diabetes, the substance comprising carnitine and an agent to promote the uptake of carnitine into the muscle tissue.

26. A substance as claimed in claim 25, in which the substance acts to increase the carnitine content of the muscle tissue.

27. A substance as claimed in claim 25, in which the substance is useful in the manufacture of a medicament for the treatment of type 2 diabetes.

28. A substance for the use in the manufacture of a medicament for the treatment of diabetes, the substance comprising carnitine and an agent to promote the uptake of carnitine into the muscle tissue, in which the substance comprises a composition as defined in claim 14.

29. A substance for use in the manufacture of a medicament for the treatment of disorders in the animal or human body caused by deficiencies in fat oxidation in muscle tissue, the substance comprising carnitine and an agent to promote the uptake of carnitine into the muscle tissue.

30. A substance for use in the manufacture of a medicament for the treatment of disorders in the animal or human body caused by deficiencies in fat oxidation in muscle tissue, the substance comprising carnitine and an agent to promote the uptake of carnitine into the muscle tissue, in which the substance comprises a composition as defined in claim 14.

31. A food supplement comprising a composition as defined in claim 14.

32. A method of treating disorders or conditions of the human and/or animal body caused by deficiencies in fat oxidation in muscle tissue, the method comprising regulating the carnitine content of the muscle tissue.

33. A method of treating disorders or conditions of the human and/or animal body caused by deficiencies in fat oxidation in muscle tissue, the method comprising regulating the carnitine content of the muscle tissue, comprising a method as defined in claim 1.

34. A method of treating disorders or conditions of the human and/or animal body caused by deficiencies in carbohydrate storage in muscle tissue, the method comprising regulating carbohydrate oxidation in the muscle tissue.

35. A method of treating disorders or conditions of the human and/or animal body caused by deficiencies in carbohydrate storage in muscle tissue, the method comprising regulating carbohydrate oxidation in the muscle tissue, comprising a method as defined in claim 1.

36. A method of treating obesity of a human or animal body, the method comprising regulating the carnitine content of the muscle tissues of the body.

37. A method of treating obesity of a human or animal body, the method comprising regulating the carnitine content of the muscle tissues of the body, comprising a method as defined in claim 1.

38. A method of treating diabetes in a human or animal body, the method comprising regulating the carnitine content of the muscle tissue in the body.

39. A method as claimed in claim 38 for the treatment of type 2 diabetes.

40. A method of treating diabetes in a human or animal body, the method comprising regulating the carnitine content of the muscle tissue in the body, comprising a method as defined in claim 1.

41.-43. (canceled)
